(19) United States
(12) Patent Application Publication
(10) Pub. No.: US 2017/0319703 A1
(43) Pub. Date: Nov. 9, 2017

(54) SHELF-STABLE MOUTH COATING GEL OR LIQUID

(71) Applicant: OraHealth Corporation, Bellevue, WA (US)

(72) Inventor: Jeffrey T. Haley, Mercer Island, WA (US)

(21) Appl. No.: 15/586,078

(22) Filed: May 3, 2017

Related U.S. Application Data
(60) Provisional application No. 62/333,241, filed on May 8, 2016.

(51) Int. Cl. A61K 47/46; A61K 47/02; A61K 35/74; A61K 31/728; A61K 31/717; A61K 47/26; A61K 31/714

(52) U.S. Cl. CPC A61K 47/46; A61K 47/26; A61K 47/02; A61K 31/714; A61K 31/728; A61K 31/717; A61K 35/74

(57) ABSTRACT

The disclosure provides dry, lipid-based mixtures comprising at least 50% by weight lipid molecules. The mixtures may comprise alkalizer molecules, non-hydrocolloidal emulsifiers and active ingredients, such as probiotic microorganisms, hyaluronan, and polyols (e.g. xylitol).

### CROSS-RELATED APPLICATIONS

This patent application claims priority from U.S. Provisional Patent Application No. 62/333,241, which was filed 8 May 2016, and incorporates the provisional application in its entirety.

### TECHNICAL FIELD

This patent application relates generally to lipid-based liquids or gels for delivering active ingredients into the mouth.

### BACKGROUND

People suffering from dry mouth need saliva substitutes. Viscous gel forms of saliva substitutes, in contrast to low viscosity liquids, are attractive because they coat better and last longer in the mouth. People suffering from gum disease, halitosis, oral mucositis, tooth decay or other diseases of the mouth need mouth coating gels or liquids to deliver ingredients for soothing, numbing, healing, reducing odor, reducing plaque, reducing tooth decay, or other benefits.

Most or all saliva substitutes and mouth coating gels contain water. Unless anti-microbial ingredients are added, any water-based mixture suitable for saliva substitution or mouth coating will grow microbes if the pH is in the range 6.8 to 8.8, and the shelf life duration will be inadequate unless antimicrobial ingredients are included. For this reason, most saliva substitutes have a pH in the range 3.5 to 6.8, which is acidic and deleterious for teeth and oral health, or they are strongly basic (alkaline) with a pH greater than 8.8 and too caustic for long term use in the mouth, or they include antimicrobials which are undesirable for long term use in the mouth.

### SUMMARY

There are claims directed to a gel or liquid mixture that is lipid-based and is soothing for human and animal mouths. In one aspect, the gel or liquid mixture comprises at least 50% by weight lipid molecules and alkalizer molecules, wherein the mixture has a water activity (aW) level not greater than 0.7. The amount of alkalizer is sufficient to give a pH of at least 7.0 when mixed with an amount of water, such as deionized water, to give an aW level of at least 0.95.

In embodiments, the alkalizer molecules are present in an amount sufficient such that when the alkalizer molecules are mixed with an amount of deionized water of pH 7.0 sufficient to give a water activity level of at least 0.95, the pH value is at least 7.0 and not great than 8.8. In other embodiments, the pH value is at least 7.0 and not greater than 8.0, or at least 7.0 and not greater than 7.6, or at least 7.1 and not greater than 7.5, or at least 7.3 and not greater than 7.5.

In embodiments, the alkalizer molecules comprise a pH buffer pair of molecule species. Examples of pH buffer pairs include mono-basic potassium phosphate and di-basic potassium phosphate, the species having a ratio to each other that buffers the pH to a value between 7.0 and 8.0 when mixed with an amount of deionized water of pH 7.0 sufficient to give a water activity level of at least 0.95.

In embodiments, at least 25% by weight of the lipid molecules are liquid at 40° C., and at least 6% by weight of the lipid molecules are solid at 40° C. In certain embodiments, the lipid molecules comprise vegetable oils.

In embodiments, the mixture further comprises at least one non-hydrocolloidal emulsifier. The emulsifier(s) may be present in a range of 0.1% to 15% of the lipid molecules by weight. In certain embodiments, the non-hydrocolloidal emulsifier molecules are selected from the group consisting of mono-and diglycerides, polysorbate, lecithin, ammonium phosphatide, egg yolk, diacetyl tartaric acid esters, sodium or calcium stearoyl-2-lactylate, sorbitan tristearate, polyglycerol esters, lactic acid esters, citric acid esters, glycerin monostearate, potassium stearate, mono propylene glyco and polyglycerol polyricinoleate. The mixture may comprise more than one emulsifier. In some mixtures, the non-hydrocolloidal emulsifier molecules comprise mono- and diglycerides that total at least 1.5% and not more than 15% of the lipid molecules by weight. In other embodiments, the non-hydrocolloidal emulsifier molecules comprise polysorbate 60 in the range of 0.1% to 2% of the lipid molecules by weight.

The mixture may further comprise an active ingredient to be delivered topically in the mouth. In embodiments, the active ingredient is at least 5% by weight polyol grains, or at least 0.2% by weight hyaluronan molecules, or at least 0.01% by weight cobalamin molecules, or at least 0.1% by weight cellulose gum molecules, or at least 0.01% by weight materials comprising one or more of calcium phosphate, casein phosphopeptide, and hydroxyapatite. In other embodiments, the active ingredient is at least ten million viable probiotic bacteria per gram of mixture.

In another aspect, the gel or liquid mixture contains a polyol. The mixture, which has a water activity level of not greater than 0.7, comprises at least 50% by weight lipid molecules and at least 5% and not greater than 30% undissolved polyol grains by weight, wherein the grain size of the polyol grains averages less than 300 microns

In embodiments, the polyol grains comprise xylitol or erythritol. The polyol may comprise at least 10% and not greater than 25% by weight.

In embodiments, at least 25% by weight of the lipid molecules are liquid at 40° C., and at least 6% by weight of the lipid molecules are solid at 40° C. In certain embodiments, the lipid molecules comprise vegetable oils.

In embodiments, the alkalizer molecules are present in an amount sufficient such that when the alkalizer molecules are mixed with an amount of deionized water of pH 7.0 sufficient to give a water activity level of at least 0.95, the pH value is at least 7.0 and not greater than 8.8. In other embodiments, the pH value is at least 7.0 and not greater than 8.0, or at least 7.0 and not greater than 7.6, or at least 7.1 and not greater than 7.5, or at least 7.3 and not greater than 7.5.

In embodiments, the alkalizer molecules comprise a pH buffer pair of molecule species. Examples of pH buffer pairs include mono-basic potassium phosphate and di-basic potassium phosphate, the species having a ratio to each other that buffers the pH to a value between 7.0 and 8.0 when mixed with an amount of deionized water of pH 7.0 sufficient to give a water activity level of at least 0.95.

In another aspect, the gel or liquid comprises probiotic microorganisms in dry, partially emulsified lipids The lipid-based mixture comprises at least 50% by weight lipid molecules; non-hydrocolloidal emulsifier molecules in a range of at least 0.1% and not greater than 15% of the lipid molecules by weight; and at least ten million viable bacteria per gram; wherein the mixture has a water activity level of not greater than 0.3.

In embodiments, at least 25% by weight of the lipid molecules are liquid at 40° C., and at least 6% by weight of the lipid molecules are solid at 40° C. In certain embodiments, the lipid molecules comprise vegetable oils.

In embodiments, at least one non-hydrocolloidal emulsifier may be present in a range of 0.1% to 15% of the lipid molecules by weight. In certain embodiments, the non-hydrocolloidal emulsifier molecules are selected from the group consisting of mono-and diglycerides, polysorbate, lecithin, ammonium phosphatide, egg yolk, diacetyl tartaric acid esters, sodium or calcium stearoyl-2-lactylate, sorbitan tristearate, polyglycerol esters, lactic acid esters, citric acid esters, glycerin monostearate, potassium stearate, mono propylene glyco and polyglycerol polyricinoleate. The mixture may comprise more than one emulsifier. In some mixtures, the non-hydrocolloidal emulsifier molecules comprise mono- and diglycerides that total at least 1.5% and not more than 15% of the lipid molecules by weight. In other embodiments, the non-hydrocolloidal emulsifier molecules comprise polysorbate 60 in the range of 0.1% to 2% of the lipid molecules by weight.

In embodiments, the mixture further comprises alkalizer molecules in an amount sufficient to give a pH at least 7.0 when the lipid-based mixture is mixed with an amount of deionized water of pH 7.0 sufficient to give a water activity level of at least 0.95. The amount of alkalizer is sufficient to give a pH of at least 7.0 when mixed with an amount of water, such as deionized water, to give an aW level of at least 0.95.

In embodiments, the alkalizer molecules are present in an amount sufficient such that when the alkalizer molecules are mixed with an amount of deionized water of pH 7.0 sufficient to give a water activity level of at least 0.95, the pH value is at least 7.0 and not great than 8.8. In other embodiments, the pH value is at least 7.0 and not greater than 8.0, or at least 7.0 and not greater than 7.6, or at least 7.1 and not greater than 7.5, or at least 7.3 and not greater than 7.5.

In embodiments, the alkalizer molecules comprise a pH buffer pair of molecule species. Examples of pH buffer pairs include mono-basic potassium phosphate and di-basic potassium phosphate, the species having a ratio to each other that buffers the pH to a value between 7.0 and 8.0 when mixed with an amount of deionized water of pH 7.0 sufficient to give a water activity level of at least 0.95.

In another aspect, a dry, lipid-based mixture that contains live probiotic microorganisms, comprises at least 50% by weight lipid molecules, wherein at least 25% by weight of the lipid molecules are liquid at 40° C. and at least 6% by weight of the lipid molecules are solid at 40° C.; and at least ten million viable bacteria per gram; wherein the mixture has a water activity level of not greater than 0.3. In embodiments, the lipid molecules comprise vegetable oils.

In embodiments, the mixture further comprises alkalizer molecules in an amount sufficient to give a pH at least 7.0 when the lipid-based mixture is mixed with an amount of deionized water of pH 7.0 sufficient to give a water activity level of at least 0.95. The amount of alkalizer is sufficient to give a pH of at least 7.0 when mixed with an amount of water, such as deionized water, to give an aW level of at least 0.95.

In embodiments, the alkalizer molecules are present in an amount sufficient such that when the alkalizer molecules are mixed with an amount of deionized water of pH 7.0 sufficient to give a water activity level of at least 0.95, the pH value is at least 7.0 and not great than 8.8. In other embodiments, the pH value is at least 7.0 and not greater than 8.0, or at least 7.0 and not greater than 7.6, or at least 7.1 and not greater than 7.5, or at least 7.3 and not greater than 7.5.

In embodiments, the alkalizer molecules comprise a pH buffer pair of molecule species. Examples of pH buffer pairs include mono-basic potassium phosphate and di-basic potassium phosphate, the species having a ratio to each other that buffers the pH to a value between 7.0 and 8.0 when mixed with an amount of deionized water of pH 7.0 sufficient to give a water activity level of at least 0.95.

In another aspect, a dry lipid-based mixture comprises hyaluronan grains. The mixture comprises at least 50% by weight lipid molecules; at least 1% and not greater than 20% hyaluronan grains by weight, wherein an average grain size of the hyaluronan grains is less than 300 microns; and wherein the mixture has a water activity level of not greater than 0.7. In some embodiments, the hyaluronan grains comprise sodium hyaluronate.

In embodiments, at least 25% by weight of the lipid molecules are liquid at 40° C., and at least 6% by weight of the lipid molecules are solid at 40° C. In certain embodiments, the lipid molecules comprise vegetable oils.

In embodiments, at least one non-hydrocolloidal emulsifier may be present in at least 0.1% and not greater than 15% of the lipid molecules by weight. In certain embodiments, the non-hydrocolloidal emulsifier molecules are selected from the group consisting of mono-and diglycerides, polysorbate, lecithin, ammonium phosphatide, egg yolk, diacetyl tartaric acid esters, sodium or calcium stearoyl-2-lactylate, sorbitan tristearate, polyglycerol esters, lactic acid esters, citric acid esters, glycerin monostearate, potassium stearate, mono propylene glyco and polyglycerol polyricinoleate. The mixture may comprise more than one emulsifier. In some mixtures, the non-hydrocolloidal emulsifier molecules comprise mono- and diglycerides that total at least 1.5% and not more than 15% of the lipid molecules by weight. In other embodiments, the non-hydrocolloidal emulsifier molecules comprise polysorbate 60 in the range of 0.1% to 2% of the lipid molecules by weight.

In other aspects, methods are provided for treating a subject, such as a human or animal, who has an oral condition by administering any of the lipid-based mixtures described herein. In embodiments, the oral condition is mucositis, dry mouth, gum disease, or halitosis.

These and other aspects of the present invention will become evident upon reference to the following detailed description.

### DETAILED DESCRIPTION

The present disclosure provides a mouth-coating liquid or gel that is shelf stable, does not grow objectionable microbes, does not damage probiotic microorganisms, and doesn't need antimicrobials. The liquid or gel composition comprises a lipid-base material that tastes and feels good and is a long-lasting lubricant, and has a low water activity (includes an insignificant amount of water). The composition may be made from partially emulsified lipids (e.g., fats or oils). Being partially emulsified with suitable emulsifiers and to a suitable degree gives an attractive mouth feel. At least half of the mixture by weight must be lipid molecules in order to result in a composition that has a good mouth feel and acceptable flavor. It may be formulated to yield a desirable pH when mixed with saliva or other source of water. The composition may comprise polyol grains, hyaluronan, or other active ingredients. The composition may be used to deliver beneficial microorganisms, for example probiotic bacteria.

### A. Lipids

The liquid or gel composition comprises at least 50% w/w lipid molecules and includes so few water molecules that it has a water activity level (aW) of less than or equal to 0.7. Long shelf-life (up to 3 yrs) may be achieved by making a gel or liquid composition with so little water that objectionable microbes will not grow. Water activity is the amount of water available for use by living organisms. A composition with substantial amounts of water can have a low water activity level if the water is tightly bound to other molecules. Water activity is measured as vapor pressure of water at the surface of the composition divided by that of pure water at the same temperature. (Pure distilled water has a water activity of exactly one.) Every microorganism has a minimum, optimum, and maximum water activity for growth. Yeast requires a water activity of at least about 0.88 for reproduction. Many bacteria, including Clostridium, require water activity of at least 0.91.

Suitable lipids include any non-toxic lipid. Lipids are classified as fatty acids, glycerolipids, glycerophospholipids, sterol lipids, sphengolipids, prenol lipids, saccharolipids, and polyketides. Their chemistry is well known. Some common types of lipids are natural fats (triglyceride), waxes, sterols, fat-soluble vitamins like vitamins A, D, E and K), mono glycerides, diglycerides and phospholipids. Typically, a suitable hydrophobic lipid is readily obtainable from commercial sources, and has a pleasant taste, e.g. vegetable oils. For best mouth feel, the hydrophobicity should be reduced with an emulsifier.

The composition may comprise a single lipid or at least two different lipids. Most often, the lipids will be from natural food sources and thus, will be a mixture of lipids. Suitable sources of lipids include vegetable oils, which contain a mixture of saturated and unsaturated fatty acids. A particular vegetable oil may be chosen for its specific mixture, because of the ratio of liquid to solid lipids at a defined temperature or health concerns or taste or other reason.

To improve mouth feel, the lipids may be partially emulsified. The extent of emulsification affects the characteristics of the final product. If there is not enough emulsifier, the mouth feel may be too oily or waxy. If there is too much emulsifier, the mouth feel may be not oily enough, and lubrication and coating effects are reduced. A suitable range for emulsifier molecules is 0.1% to 15% of the lipid molecules by weight. Examples include mono- and di-glycerides in the range of 1.5% to 15% of the lipid molecules by weight and polysorbate 60 in the range of 0.1% to 2% of the lipid molecules by weight.

The composition should not comprise more than 1% hydrocolloids, because, although hydrocolloids work as emulsifiers for lipids/water, they absorb too much water from saliva and make a swollen blob that has an unattractive mouth feel. Suitable non-hydrocolloidal emulsifiers include mono- and diglycerides, polysorbate, lecithin, ammonium phosphatide, egg yolk, diacetyl tartaric acid esters, sodium or calcium stearoyl-2-lactylate, sorbitan tristearate, polyglycerol esters, lactic acid esters, citric acid esters, glycerin monostearate, potassium stearate, mono propylene glycol, and polyglycerol polyricinoleate. More than one emulsifier may be used in combination as long as the total amount is within 0.1% to 15% of the lipid molecules by weight.

Adjusting the ratio of liquid to solid lipids affects the viscosity of the composition. To make a gel, (which means a thick substance—due to high viscosity), the lipids have a desired ratio of liquids to solids at human or animal mouth temperatures as measured at particular temperature. For gel embodiments, preferred viscosity is 500,000 to 2,000,000 centipoise. A standard temperature for rating the liquid to solid ratio in the lipids industry is 40° C., which is close to the temperature of a human mouth. A suitable ratio of liquid to solid is at least 25% by weight of the lipid molecules being liquid at 40° C. and at least 6% by weight of the lipid molecules being solid at 40° C. If the lipid material is too liquid, it dissipates too quickly in the mouth. If the lipid material is too solid, it dissipates too slowly and has an unattractive mouth feel. Thus, the liquid fraction of the lipids at 40 ° C. can be in the range 6% to 75% and the solid fraction can be in the range 25% to 94%.

### B. PH Control

In one aspect, to maintain a desired pH level regardless of additives to the compositions, pH buffers may be incorporated that hold the pH within a narrow range that is neutral or slightly alkaline.

Low pH of materials in the mouth (below 6.8) creates an acidic environment and promotes the growth of aciduric bacteria, creating an inhospitable environment for protective oral bacteria. This allows a shift in the oral environmental balance to favor cariogenic bacteria, which further lowers the salivary pH and the cycle continues. Cariogenic bacteria thrive in an acidic environment and cause tooth decay. Furthermore, periodontal diseases in humans and other mammals are predominantly associated with Gram-negative anaerobic organisms. Likely agents of periodontal disease include Porphyromonas gingivalis, Fusobacterium nucleatum and Prevotella intermedia. These bacteria grow at slightly acidic pH (P. gingivalis grows at a pH of 6.5-7.0, P. intermedia grows at a pH of 5.0-7.0, and F. nucleatum grows at a pH of 5.5-7.0). Thus, placing in the mouth any material that lowers the pH to 7.0 or lower is likely to promote periodontal disease.

Periodontal disease is initiated by plaque on the roots of teeth and plaque also causes tooth decay. The two key factors for dental plaque formation are: (1) there must be oral bacteria to attack food particles and (2) the pH must elevate above 7.6 to grow the plaque.

pH effects in materials that linger in a human mouth

| | | | | |
|---|---|---|---|---|
| pH <6.8 | pH 6.8-7.0 | pH 7.0-7.6 | pH 7.6-8.8 | pH >8.8 |

| | | |
|---|---|---|
| OK shelf life | Shelf life too short if water activity >7.0 | OK shelf life |

| | | |
|---|---|---|
| Bad for tooth decay and | Neutral range | Bad for plaque |
| periodontal disease | for human | formation, leading |
| | mouths | to tooth decay and |
| | | periodontal disease |

In embodiments, the pH of the composition is in the range 7.0 to 8.8, or 7.0 to 8.0, or 7.0 to 7.6, or 7.1 to 7.5, or 7.3 to 7.5. To achieve the desired pH, weak alkalizer molecules, such as calcium carbonate (CaCO₃) or potassium bicarbonate (KHCO₃), can be included in a suitable amount to result in a pH of 7.0 to 8.8, or 7.0-8.0, or 7.0-7.6, or 7.1 to 7.5, or 7.3 to 7.5 when placed in a mouth with saliva. To determine a suitable amount of alkalizer, deionized water (pH 7.0) is added to compositions comprising varying amounts of alkalizer to give a water activity level of 0.95 or higher, and the pH is measured. The alkalizer should not be so strong that it creates temporary local spots with a pH above about 10 as the alkalizer mixes with water of saliva. In addition, the alkalizer particles are typically finely ground to avoid adding a gritty feel.

In certain embodiments, the alkalizer can be a combination of pH buffering molecule species such that, when the molecules dissolve in the water of saliva, they buffer the saliva to within a narrow pH range even when mixed with modest amounts of more acid or alkaline ingredients. One suitable pH buffer pair is mono-basic potassium phosphate and di-basic potassium phosphate. This pH buffer pair can be included in a ratio to each other that buffers the pH to a desired value between about 7.0 and about 7.6, or about 7.3 to about 7.5, when the gel is mixed with enough water to raise the water activity level to 0.95 or higher. Examples of other suitable buffering pairs are Sørensen's phosphate buffer, NaH₂PO₄—Na₂HPO_{4,} and aphosphate/citrate buffer of dibasic sodium phosphate and citric acid. Other suitable buffers are well known.

pH control as described herein may be used in combination with delivery of active or desired ingredients, such as those described below, e.g., polyols, hyaluronan, and probiotic microorganisms, calcium, phosphorus, amorphous calcium phosphate, hydroxyapatite, hyaluronan, cobalamin, steroids, NSAIDs, antibiotics, or any other ingredient.

### C. Polyols

The lipid-based, liquid or gel compositions can be used to deliver ingredients that stimulate saliva via flavor, such as sweet flavor (xylitol, erythritol, other polyols, sucralose, stevia, and other non-cariogenic sweeteners) or savory flavor such as food flavors made by yeasts or vanilla or vanillin. Of course, any other flavor can also be delivered, but in general, sour flavors, salty flavors and bitter flavors are not used, because of deleterious characteristics (e.g. acidic, bitter).

Dry mouth is a cause of or contributes to tooth decay. Retention of xylitol or erythritol or other polyols in the mouth reduces plaque and tooth decay. By delivering xylitol or another polyol to the mouth in a lipid-based gel to keep water activity levels low, shelf life is lengthened because microbial growth is reduced. Polyols do not dissolve in lipids however.

The lipid-based compositions can be used to deliver xylitol, erythritol or other polyols in the mouth, which reduce plaque and tooth decay and stimulate saliva. Because polyols are not lipid-soluble, the xylitol or other polyol is added as a fine powder or very small granules (typically diameter smaller than 0.3 millimeter (300 microns)) so that the grains are held in suspension. Usually, the polyols are suspended in a gel that can be placed in the mouth as a blob. Then, as the gel slowly erodes in the mouth, the polyol grains are slowly exposed to saliva and dissolve, providing stimulation of more saliva via its sweet flavor. This slow release of the polyol can improve effectiveness for reducing tooth decay because a concentration of polyols around the teeth is maintained over time, particularly when the gel is pushed into gaps between the teeth and/or orthodontic braces.

### D. Probiotic Bacteria

The compositions can also comprise desired additional ingredients such as probiotic bacteria. Delivering live, probiotic species of bacteria benefits oral health, such as reducing gum disease and halitosis. For adequate shelf life, useful species of bacteria need to be kept dry until placed in the mouth, and therefore cannot be provided in water-based gels or liquids.

The lipid-based gel or liquid can be used to deliver probiotic organisms, usually bacteria, in the mouth to promote oral health and healing. The probiotic organisms may be, for example, Lactobacillus spp. (e.g., L. acidophilus, L. reuteri), Bifidobacterium spp. (e.g., B. bifidum, B. longuml), Streptococcus. salivarius K12 or M18. Bacillus coagulans or other helpful microorganisms (see, Fijan, Int. J. Environ. Res. Public Health, 11:4745, 2014). The microorganisms are generally added as a fine powder or very small granules (smaller than 0.3 mm (300 microns)) so that the grains are held in suspension. Typically, the compositions comprise at least ten million viable organisms per gram of gel or liquid. A mixture of species may also be used. The gel can be placed in the mouth as a blob. Then, as the gel slowly erodes in the mouth, the probiotic bacteria are slowly exposed to saliva and become active.

### E. Hyaluronan

When the mouth lining is sensitive to irritation, such as from dry mouth or mucositis, a water-based gel can be soothing. Hyaluronan forms an effective gel for this purpose, and hyaluronan is a healing agent. But such gels have a short shelf life when they have a pH in the neutral range of 7.0 to 7.6. The gel can be formed by adding dry powder to saliva in the mouth but this is awkward and inconvenient and creates an unattractive mouth feel.

The lipid-based gel or liquid composition can be used to deliver hyaluronan in the mouth, in order to coat and soothe the mouth and promote healing. The hyaluronan, which may be, for example, sodium hyaluronate or another form of hyaluronic acid, is added as a fine powder or very small granules (smaller than 0.3 mm (300 microns)) such that the grains are held in suspension. A typical range of hyaluronan is 0.1% to 10% hyaluronan grains by weight. The gel can be placed in the mouth as a blob. Then, as the gel slowly erodes in the mouth, the hyaluronan grains are slowly exposed to saliva and dissolve.

### F. Method of Manufacturing

The gels or liquid compositions are made by mixing the ingredients together with any type of mixer, such as a bakery-type vertical mixer with an orbital rotating paddle such as a Hobart.

For treatment of oral conditions, the gel or liquid may be supplied in any container. Generally, the container will have a low oxygen transmission rate and be impervious to water and water vapor transmission, especially when the gel or liquid contains a hygroscopic ingredient. In one embodiment, the gel or liquid is supplied from a collapsible tube from which one squirts directly into the mouth and uses the tongue to spread to all surfaces of the mouth. The liquid embodiment may be placed in a spray bottle or sipping bottle.

### G. Methods of Use

The lipid-based compositions may be used to deliver ingredients to treat, remedy, reduce, or prevent topically addressable oral conditions such as dry mouth, caries, gingivitis, periodontitis, halitosis, demineralization of tooth enamel or roots, mucositis, mouth ulcers, lichen planus, yeast or fungal infection, oral cancer, and topical oral pain.

For relief of dry mouth, the gel composition is a helpful lubricant and can stimulate saliva with the addition of flavors, such as sweet or savory. Sweet flavor may be included in many forms, including xylitol, erythritol, and other non-cariogenic sweeteners. The amount of flavor will typically be determined in part by individual or population preference for taste and amount known to stimulate saliva. Generally, flavor is present in a range of 1% to 30% depending on the strength of the flavoring. When savory flavor is to be included, it may be vanilla or vanillin. Peppermint oil may be used. More than one flavor may be included.

For caries, such as caused by dry mouth, the gel or liquid composition may include caries-suppressing ingredients, such as xylitol, erythritol, other polyols, fluoride, chlorhexidine, or triclosan. When xylitol is used, typically it is present at a range of 10% to 25% by weight. The amount of other caries-suppressing ingredients may be guided by knowledge in the art or determined by clinical trials.

For teeth demineralization, the gel or liquid may include substances that promote remineralization such as calcium phosphate, casein phosphopeptide, or hydroxyapatite.

For mucositis, including mouth ulcers, the gel or liquid may include fine particle hyaluronan in the range of 0.1% to 10%, or in the range of 0.2% to 3% by weight; or cobalamin such a methyl-cobalamin or cyano-cobalamin, typically in the range 0.01% to 0.2% by weight. The gel or liquid may comprise both hyaluronan and cobalamin.

For lichen planus and other topical diseases of the mouth, the gel or liquid may include one or more steroids or NSAIDs.

For yeast or other fungal infections, the gel or liquid may include one or more anti-fungal ingredients. Examples of anti-fungal ingredients include clotrimazole, econazole, miconazole, terbinafine, fluconazole, ketoconazole, and amphotericin.

For oral pain, the gel or liquid may include one or more of benzocaine, lidocaine or other anesthetics.

For oral cancer, the gel or liquid may include topical anti-cancer drugs.

For halitosis, gingivitis and periodontitis, the gel or liquid may include probiotic bacteria, anti-plaque ingredients or antibiotics. Suitable species of probiotic bacteria for both gum disease and halitosis include Lactobacillus reuteri, Lactobacillus salivarius, and Lactobacillus brevis, as well as bifidobacteria.

The preferred usage is enough to keep a thin coating of the gel or liquid on mouth lining surfaces as many hours per day as possible. When applied to the lips, the gel or liquid can also provide effective relief for dry lips.

Alternatively, the gel may be squished between the teeth and left surrounding the base of each tooth, slowly releasing the active ingredient(s), e.g., xylitol or probiotic bacteria. This can be most effective when done at bedtime when saliva flow is lowest to maximize the duration of release and lingering of the active ingredients.

Some users may find it helpful to place a blob of gel in the lower lip or lower cheek or upper cheek from where the gel can slowly emerge overtime to prolong the benefits of the gel. This may be especially helpful while sleeping to reduce the need to replenish the gel while sleeping. People with orthodontic braces can squish the gel into niches in their braces which form little reservoirs from which the gel spreads out over time, especially while sleeping.

The following examples are offered by way of illustration, and not by way of limitation.

### EXAMPLES

### Example 1

### Lipid-Based Composition with Water Activity 0.6

This example presents exemplary formulae for a lipid-based composition with a water activity less than 0.7.

| | grams | % |
|---|---|---|
| Water | 117 | 1.9% |
| mono-basic Potassium (K) PO4 | 12.01 | 0.19% |
| di-basic Potassium (K) PO4 | 84.72 | 1.34% |
| Blend of hydrogenated oils and emulsifiers | 4902 | 77.3% |
| Canola oil | 258 | 4.1% |
| xylitol - fine grain | 1176 | 18.5% |
| vanillin | 5.9 | 0.1% |
| | 6342 | |

Mix 4-6 minutes.

| | Percent |
|---|---|
| Ingredient | by weight |
| blend of hydrogenated oils and | 76.5% |
| emulsifiers | |
| canola oil | 4.3% |
| vanillin | 0.11% |
| mono-basic Potassium (K) PO4 | 0.19% |
| di-basic Potassium (K) PO4 | 1.32% |
| xylitol - fine grain | 17.6% |

From the foregoing it will be appreciated that, although specific embodiments have been described herein for purposes of illustration, various modifications may be made without deviating from the spirit and scope of the invention. Accordingly, the invention is not limited except as by the appended claims.

1. A lipid-based mixture that is soothing for human and animal mouths, comprising:
(a) at least 50% by weight lipid molecules; and
(b) alkalizer molecules in an amount sufficient to give a pH of at least 7.0 when mixed with an amount of deionized water of pH 7.0 sufficient to give a water activity level of at least 0.95;
and wherein the mixture has a water activity level not greater than 0.7.

2. The mixture of claim 1, wherein at least 25% by weight of the lipid molecules are liquid at 40° C., and at least 6% by weight of the lipid molecules are solid at 40° C.

3. The mixture of claim 1, wherein the lipid molecules comprise vegetable oils.

4. The mixture of claim 1, further comprising:
(c) non-hydrocolloidal emulsifier molecules in a range of at least 0.1% and not greater than 15% of the lipid molecules by weight.

5. The mixture of claim 4 ,wherein the non-hydrocolloidal emulsifier molecules are selected from the group consisting of mono-and diglycerides, polysorbate, lecithin, ammonium phosphatide, egg yolk, diacetyl tartaric acid esters, sodium or calcium stearoyl-2-lactylate, sorbitan tristearate, polyglycerol esters, lactic acid esters, citric acid esters, glycerin monostearate, potassium stearate, mono propylene glyco and polyglycerol polyricinoleate.

6. The mixture of claim 4 ,wherein the non-hydrocolloidal emulsifier molecules comprise mono- and diglycerides in the range of 1.5% to 15% of the lipid molecules by weight.

7. The mixture of claim 4, wherein the non-hydrocolloidal emulsifier molecules comprise polysorbate 60 in the range of 0.1% to 2% of the lipid molecules by weight.

8. The mixture of claim 1, wherein when the alkalizer molecules are mixed with an amount of deionized water of pH 7.0 sufficient to give a water activity level of at least 0.95, the pH value is at least 7.0 and not greater than 8.8.

9. The mixture of claim 8, wherein the alkalizer molecules comprise a pH buffer pair of molecule species.

10. The mixture of claim 9, wherein the pH buffer pair of molecule species is mono-basic potassium phosphate and di-basic potassium phosphate, the species having a ratio to each other that buffers the pH to a value at least 7.0 and not greater than 8.0 when mixed with an amount of deionized water of pH 7.0 sufficient to give a water activity level of at least 0.95.

11. The mixture of claim 1 further comprising:
(d) an active ingredient to be delivered topically in the mouth.

12. The mixture of claim 11, wherein the active ingredient is at least 5% by weight polyol grains.

13. The mixture of claim 11, wherein the active ingredient is at least 0.2% by weight hyaluronan molecules.

14. The mixture of claim 11, wherein the active ingredient is at least 0.01% by weight cobalamin molecules.

15. The mixture of claim 11 wherein the active ingredient is at least 0.1% by weight cellulose gum molecules.

16. The mixture of claim 11, wherein the active ingredient is at least 0.01% by weight materials comprising one or more of calcium phosphate, casein phosphopeptide, and hydroxyapatite.

17. The mixture of claim 11, wherein the active ingredient is at least ten million viable bacteria per gram of mixture.

18. A lipid-based mixture containing a polyol comprising:
(a) at least 50% by weight lipid molecules;
(b) at least 5% and not greater than 30% undissolved polyol grains by weight,
wherein average grain size of the polyol grains is less than 300 microns; and
wherein the mixture has a water activity level of not greater than 0.7.

19. The mixture of claim 18, wherein the polyol grains comprise xylitol.

20. The mixture of claim 18, wherein the polyol grains comprise erythritol.

21. The mixture of claim 18, wherein the polyol comprises at least 10% and not greater than 25% by weight.

22. The mixture of claim 18, wherein the lipid molecules comprise vegetable oils.

23. The mixture of claim 18, further comprising alkalizer molecules in an amount sufficient to give a pH at least 7.0 when the lipid-based mixture is mixed with an amount of deionized water of pH 7.0 sufficient to give a water activity level of at least 0.95;

24.-41. (canceled)
